(19) United States
(12) Patent Application Publication
(10) Pub. No.: US 2026/0284131 A1
(43) Pub. Date: Sep. 24, 2026

(54) Compositions and Methods for Increasing Urination

(71) Applicant: YUJIAN International Biotechnology, Inc., Hsin-Chu (TW)

(72) Inventors: Yao-Jen TU, Hsin-Chu (TW); Yu-Shan LIU, Hsin-Chu (TW); Yu-Ting KU, Hsin-Chu (TW); Cheng-Hao CHEN, Hsin-Chu (TW); Fong-Je LIN, Hsin-Chu (TW); Chih-Yuan CHENG, Hsin-Chu (TW)

(21) Appl. No.: 19/243,013

(22) Filed: Jun. 19, 2025

(30) Foreign Application Priority Data
Mar. 20, 2025 (TW) 114110586

(51) Int. Cl. A61K 36/23; A61K 31/122; A61K 36/05; A61K 36/235; A61K 36/28; A61K 36/288; A61K 36/48; A61K 36/73; A61K 36/77; A61K 36/87; A61K 36/8994; A61P 13/02

(52) U.S. Cl. CPC A61K 36/23; A61K 31/122; A61K 36/05; A61K 36/235; A61K 36/28; A61K 36/288; A61K 36/48; A61K 36/73; A61K 36/77; A61K 36/87; A61K 36/8994; A61P 13/02; A61K 2236/331; A61K 2236/51

(57) ABSTRACT

The present disclosure concerns compositions and methods for increasing urination. The composition comprises astaxanthin and herbal extracts from a variety of plants, including Apiaceae Carum carvi, Spiraea ulmaria, Paullinia cupana, Solidago virgaurea, Foeniculum vulgare Mill, Taraxacum officinale Weber, and red bean. The astaxanthin can be obtained from Haematococcus pluvialis. The present disclosure contemplates that combinations of the above-identified components at a specific range of percentages can effectively facilitate urination.

### CROSS-REFERENCE TO RELATED APPLICATIONS

This application claims priority to co-pending Taiwan patent application No. 114110586, filed on Mar. 20, 2025, titled “Compositions and Methods for Facilitating Urination,” which is incorporated herein by reference for its entirety.

### TECHNICAL FIELD

The present disclosure is related to compositions and methods for increasing urination, particularly to a composition prepared by using natural botanical extracts.

### BACKGROUND

Edema generally refers to the retention of water within an organism that accumulates in the interstitial tissues or under the skin, resulting in swelling and loss of elasticity. Usually, edema is related to poor water metabolism in the body. Common causes of it include: remaining in the same position for long periods (e.g., prolonged sitting or standing), excessive sodium intake (such as having a high-salt diet), insufficient protein intake, the female menstrual cycle, side effects of medications, or diseases (e.g., lymphatic obstruction, thyroid dysfunction, kidney disease, heart disease, liver disease, etc.). Edema caused by medications or diseases requires treatment for relief, while in most situations, edema caused by lifestyle habits can be improved through good dietary and lifestyle habits until medication becomes necessary. However, modern lifestyles are often the main cause of non-disease-related edema. Therefore, there is a need in the field for a nutritional supplement that promotes urination to help effectively prevent and improve edema under modern lifestyle conditions without relying on medication.

### BRIEF SUMMARY

In one aspect, the present disclosure is related to a composition for increasing urination, comprising: 1 to 10 wt % of a botanical extract, which comprises an Apiaceae Carum carvi extract, a Spiraea ulmaria extract, a Paullinia cupana extract, a Solidago virgaurea extract, a Foeniculum vulgare Mill extract, and a Taraxacum officinale Weber extract; 0.1 to 1 wt % of a Haematococcus pluvialis extract; and 0.1 to 1 wt % of a red bean extract.

In another aspect, the present disclosure is related to a composition for increasing urination, comprising 1 to 10 wt % of a botanical extract, which comprises an Apiaceae Carum carvi extract, a Spiraea ulmaria extract, a Paullinia cupana extract, a Solidago virgaurea extract, a Foeniculum vulgare Mill extract, and a Taraxacum officinale Weber extract; 0.001 to 0.1 wt % of astaxanthin; and 0.1 to 1 wt % of a red bean extract, wherein the astaxanthin is purified from Haematococcus pluvialis, Adonis aestivalis, Euphasia supurba, salmon, red snapper, or a mixture thereof.

Yet in another aspect, the present disclosure is related to a method for increasing urination, comprising administering an effective amount of a composition of the present disclosure to a subject.

### BRIEF DESCRIPTION OF THE SEVERAL VIEW OF THE DRAWING

FIG. 1 is a graphic presentation showing the benchmark urination of the control-group rats and the experiment-group rats (the first phase of the experiment). The control-group rats and the experiment-group rats did not show a significant difference in statistics (p>0.05) during Hours 0 to 6, Hours 6 to 24, and Hours 0 to 24.

FIG. 2 is a graphic presentation showing the urination of the control-group rats and the experiment-group rats (the second phase of the experiment). The experiment-group rats had significantly higher urination (*p=0.017) during Hours 6 to 24. During Hours 0 to 6 and Hours 0 to 24, the increase in urination observed in the experiment-group rat was even more significant (**p=0.0001).

FIG. 3A is a photo showing the bladder of a control-group rat (indicated by an arrow). No visible lesions were observed.

FIG. 3B is a photo showing the bladder of an experiment-group rat (indicated by an arrow). No visible lesions were observed.

### DETAILED DESCRIPTION

Edema is a relatively common health condition. Although it has many causes, most cases are temporary and simply due to prolonged periods of maintaining the same posture or poor water metabolism efficiency associated with the female menstrual cycle. This situation is especially common in the morning as the metabolic rate usually slows down during sleep. Such non-disease-related edema usually can be improved by adjusting the patient's daily routines without the need for medication. Nevertheless, such a situation can be more effectively addressed with a proper dietary therapy to promote urination and accelerate water metabolism, thereby avoiding the continued accumulation of fluid and the potential development of other health concerns.

However, despite the fact that some food ingredients have been known to have potential benefits on increasing urination, achieving a desired improvement from food intake is actually difficult. This is because obtaining sufficient amounts of diuretic components from diet requires consuming a large quantity of such foods, which may be impractical. Besides, many of these so-called diuretic foods may never have been scientifically validated through experimentation. Therefore, the present disclosure combines natural botanical extracts and components in specific proportions, and, after experimental verification of their diuretic effect, contemplates the composition and methods of using it.

### Compositions of the Present Disclosure

In one aspect, the present disclosure provides a composition for increasing urination, comprising: 1 to 10 wt % of a botanical extract, which comprises an Apiaceae Carum carvi extract, a Spiraea ulmaria extract, a Paullinia cupana extract, a Solidago virgaurea extract, a Foeniculum vulgare Mill extract, and a Taraxacum officinale Weber extract; 0.1 to 1 wt % of a Haematococcus pluvialis extract; and 0.1 to 1 wt % of a red bean extract. In another aspect, the present disclosure provides a composition for increasing urination, comprising 1 to 10 wt % of a botanical extract, which comprises an Apiaceae Carum carvi extract, a Spiraea ulmaria extract, a Paullinia cupana extract, a Solidago virgaurea extract, a Foeniculum vulgare Mill extract, and a Taraxacum officinale Weber extract; 0.001 to 0.1 wt % of astaxanthin; and 0.1 to 1 wt % of a red bean extract. As described herein, “increasing urination” means promoting the urination in volume and/or frequency. Without wishing to be bound by any theory, the present disclosure believes that increasing urination is positively correlated with the alleviation of edema symptoms. Therefore, in some embodiments, the composition for increasing urination of the present disclosure can also be used to improve edema. In other embodiments, “increase urination” as used in the present disclosure is not limited to whether an edema condition is improved. In some specific embodiments, “increasing urination” can be determined by measuring the volume and frequency of urination before and after administering the composition of the present disclosure.

In some specific embodiments, the present disclosure provides a composition for increasing urination, which substantially consists of 1 to 10 wt % of a botanical extract, which comprises an Apiaceae Carum carvi extract, a Spiraea ulmaria extract, a Paullinia cupana extract, a Solidago virgaurea extract, a Foeniculum vulgare Mill extract, and a Taraxacum officinale Weber extract; 0.1 to 1 wt % of a Haematococcus pluvialis extract; and 0.1 to 1 wt % of a red bean extract. In other specific embodiments, the present disclosure provides a composition for increasing urination, which substantially consists of 1 to 10 wt % of a botanical extract, which comprises an Apiaceae Carum carvi extract, a Spiraea ulmaria extract, a Paullinia cupana extract, a Solidago virgaurea extract, a Foeniculum vulgare Mill extract, and a Taraxacum officinale Weber extract; 0.001 to 0.1 wt % of astaxanthin; and 0.1 to 1 wt % of a red bean extract. “Substantially consist of,” as used herein, refers to situations where the composition does not comprise another diuretic or urination-increasing component, but the composition is not excluded from having components of other functions. For example, the composition might further comprise non-active ingredients, such as a carrier, binder, diluent, disintegrant, filler, glidant, lubricant, coloring agent, preservative, sweetener, surfactant, solvent, coating agent, or a mixture thereof. In some embodiments, the non-active ingredients might comprise at least 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, 80 wt %, 85 wt %, 90 wt %, 95 wt %, 98 wt %, 99 wt %, or any range defined by two foregoing endpoints, such as: 10 to 99 wt %, 10 to 98 wt %, 10 to 95 wt %, 10 to 90 wt %, 10 to 80 wt %, 10 to 70 wt %, 10 to 60 wt %, 10 to 50 wt %, 10 to 40 wt %, 10 to 30 wt %, 10 to 20 wt %, 30 to 99 wt %, 30 to 98 wt %, 30 to 95 wt %, 30 to 90 wt %, 30 to 80 wt %, 30 to 70 wt %, 30 to 60 wt %, 30 to 50 wt %, 30 to 40 wt %, 30 to 30 wt %, 50 to 99 wt %, 50 to 98 wt %, 50 to 95 wt %, 50 to 90 wt %, 50 to 80 wt %, 50 to 70 wt %, 50 to 60 wt %, 70 to 99 wt %, 70 to 98 wt %, 70 to 95 wt %, 70 to 90 wt %, or 70 to 80 wt %.

Astaxanthin. Astaxanthin is a natural lipid-soluble red pigment, which is a kind of keto-carotenoid having an IUPAC name, 3,3′-Dihydroxy-β,β-carotene-4,4′-dione. The structure of astaxanthin contains hydroxyl and ketone groups extending to a long carbon chain. The long carbon chain and the hydroxyl group provide astaxanthin with an amphiphilic nature, and the length of the long carbon chain suffice and enables the astaxanthin molecule across cell membranes; therefore, the astaxanthin molecule is able to exert anti-oxidation within and outside of a cell. Astaxanthin was first discovered and isolated from lobster shells, and then scientists subsequently identified the presence thereof in other organisms, such as Haematococcus pluvialis, Adonis aestivalis, Euphasia supurba, salmon, red snapper, etc. Among them, Haematococcus pluvialis is known to have the highest amount of astaxanthin. In some embodiments, the source of the astaxanthin of the present disclosure's composition is not limited; while in some specific embodiments, the astaxanthin is isolated or purified from Haematococcus pluvialis or Antarctic krill (Euphausia superba).

In many embodiments, the astaxanthin of the present disclosure's composition is purified from Haematococcus pluvialis; or, the present disclosure's composition comprises a Haematococcus pluvialis extract to provide the astaxanthin. In some embodiments, the Haematococcus pluvialis extract is a supercritical carbon dioxide extract. In some specific embodiments, the Haematococcus pluvialis extract is prepared by using a method comprising: obtaining a Haematococcus pluvialis, drying the Haematococcus pluvialis, and extracting the dried Haematococcus pluvialis using supercritical carbon dioxide to obtain the Haematococcus pluvialis extract. In many embodiments, the drying is performed using freeze-drying (or, lyophilization). In many specific embodiments, the composition of the present disclosure comprises at least about 0.1, 0.2, 0.25, 0.3, 0.35, 0.4, 0.5, 0.55, 0.6, 0.65, 0.7, 0.8, 0.9, or 1 wt % of the Haematococcus pluvialis extract, or any range defined by two foregoing endpoints, such as: 0.1 to 1 wt %, 0.1 to 0.8 wt %, 0.1 to 0.6 wt %, 0.1 to 0.5 wt %, 0.1 to 0.3 wt %, 0.2 to 1 wt %, 0.2 to 0.8 wt %, 0.2 to 0.6 wt %, 0.2 to 0.5 wt %, 0.2 to 0.3 wt %, 0.25 to 1 wt %, 0.25 to 0.8 wt %, 0.25 to 0.6 wt %, 0.25 to 0.5 wt %, or 0.25 to 0.3 wt %.

In some embodiments, the present disclosure's composition comprises at least about 0.001, 0.002, 0.003, 0.005, 0.008, 0.01, 0.02, 0.03, 0.05, 0.06, 0.07, 0.08, or 0.1 wt % of Astaxanthin, or any range defined by two foregoing endpoints, such as: 0.001 to 0.1 wt %, 0.001 to 0.08 wt %, 0.001 to 0.07 wt %, 0.001 to 0.06 wt %, 0.001 to 0.05 wt %, 0.001 to 0.01 wt %, 0.001 to 0.007 wt %, 0.001 to 0.005 wt %, 0.002 to 0.1 wt %, 0.002 to 0.08 wt %, 0.002 to 0.07 wt %, 0.002 to 0.06 wt %, 0.002 to 0.05 wt %, 0.002 to 0.01 wt %, 0.002 to 0.007 wt %, 0.002 to 0.005 wt %, 0.01 to 0.1 wt %, 0.01 to 0.08 wt %, 0.01 to 0.07 wt %, 0.01 to 0.06 wt %, 0.01 to 0.05 wt %, 0.02 to 0.1 wt %, 0.02 to 0.08 wt %, 0.02 to 0.07 wt %, 0.02 to 0.06 wt %, or 0.02 to 0.05 wt %.

Depending on the optical activity of the hydroxyl groups at both ends of the astaxanthin structure, astaxanthin has left-handed (3S-3S′), racemic (3R-3S′), and right-handed (3R-3R′) structures. In some embodiments, the composition of the present disclosure comprises left-handed astaxanthin, racemic astaxanthin, and/or right-handed astaxanthin. In some embodiments, the astaxanthin of the present disclosure's composition comprises about 20 mol %, 25 mol %, 30 mol %, 35 mol %, 40 mol %, 45 mol %, 50 mol %, 55 mol %, 60 mol %, 65 mol %, 70 mol %, 75 mol %, 80 mol %, 85 mol %, 90 mol %, 95 mol %, or 100 mol % of left-handed astaxanthin, or any range defined by the foregoing two values, such as, 20 mol % to 100 mol %, 20 mol % to 95 mol %, 20 mol % to 90 mol %, 20 mol % to 85 mol %, 20 mol % to 80 mol %, 20 mol % to 75 mol %, 20 mol % to 70 mol %, 20 mol % to 65 mol %, 20 mol % to 60 mol %, 20 mol % to 55 mol %, 20 mol % to 50 mol %, 20 mol % to 45 mol %, 20 mol % to 40 mol %, 20 mol % to 35 mol %, 20 mol % to 30 mol %, 20 mol % to 25 mol %, 30 mol % to 100 mol %, 30 mol % to 95 mol %, 30 mol % to 90 mol %, 30 mol % to 85 mol %, 30 mol % to 80 mol %, 30 mol % to 75 mol %, 30 mol % to 70 mol %, 30 mol % to 65 mol %, 30 mol % to 60 mol %, 30 mol % to 55 mol %, 30 mol % to 50 mol %, 30 mol % to 45 mol %, 30 mol % to 40 mol %, 30 mol % to 35 mol %, 40 mol % to 100 mol %, 40 mol % to 95 mol %, 40 mol % to 90 mol %, 40 mol % to 85 mol %, 40 mol % to 80 mol %, 40 mol % to 75 mol %, 40 mol % to 70 mol %, 40 mol % to 65 mol %, 40 mol % to 60 mol %, 40 mol % to 55 mol %, 40 mol % to 50 mol %, 40 mol % to 45 mol %, 50 mol % to 100 mol %, 50 mol % to 95 mol %, 50 mol % to 90 mol %, 50 mol % to 85 mol %, 50 mol % to 80 mol %, 50 mol % to 75 mol %, 50 mol % to 70 mol %, 50 mol % to 65 mol %, 50 mol % to 60 mol %, 50 mol % to 55 mol %, 60 mol % to 100 mol %, 60 mol % to 95 mol %, 60 mol % to 90 mol %, 60 mol % to 85 mol %, 60 mol % to 80 mol %, 60 mol % to 75 mol %, 60 mol % to 70 mol %, 60 mol % to 65 mol %, 70 mol % to 100 mol %, 70 mol % to 95 mol %, 70 mol % to 90 mol %, 70 mol % to 85 mol %, 70 mol % to 80 mol %, 70 mol % to 75 mol %, 80 mol % to 100 mol %, 80 mol % to 95 mol %, 80 mol % to 90 mol %, 80 mol % to 85 mol %, 90 mol % to 100 mol %, 90 mol % to 95 mol %, or 95 mol % to 100 mol % of left-handed astaxanthin.

Red bean extract. As described above, the composition of the present disclosure comprises a red bean extract, for example, a red bean seed coat extract. In many embodiments, the composition of the preset disclosure comprises at least about 0.1, 0.2, 0.3, 0.4, 0.5, 0.6, 0.7, 0.8, 0.9, or 1 wt %, or any range defined by two foregoing endpoints, such as: 0.1 to 1 wt %, 0.1 to 0.9 wt %, 0.1 to 0.8 wt %, 0.1 to 0.7 wt %, 0.1 to 0.6 wt %, 0.1 to 0.5 wt %, 0.1 to 0.3 wt %, 0.2 to 1 wt %, 0.2 to 0.9 wt %, 0.2 to 0.8 wt %, 0.2 to 0.7 wt %, 0.2 to 0.6 wt %, 0.2 to 0.5 wt %, 0.2 to 0.3 wt %, 0.4 to 1 wt %, 0.4 to 0.9 wt %, 0.4 to 0.8 wt %, 0.4 to 0.7 wt %, 0.4 to 0.6 wt %, or 0.4 to 0.5 wt %.

In many embodiments, the red bean extract is prepared by a method comprising: mixing a red bean with water to obtain a mixture, heating the mixture of the red bean and the water, and drying the heated mixture, thereby obtaining the red bean extract. In many specific embodiments, before heating, the method comprises filtering the mixture to remove solid substances therein. In many specific embodiments, the heating is performed at 90 to 120 degrees Celsius, 100 to 120 degrees Celsius, or 90 to 110 degrees Celsius for 10 to 60, 10 to 50, 30 to 60, or 30 to 50 minutes. For instance, the heating is performed at 110 degrees Celsius for 40 minutes. The drying can be performed by using spray drying, freeze-drying, or vacuum drying. In some embodiments, before drying the mixture, the method further comprises condensing the mixture. Said condensing means reducing the volume of the mixture by at least about 10%, 20%, 30%, 40%, 50%, 60%, 70%, 80%, or 90%, which can be achieved by reducing the water content of the mixture.

Without wishing to be bound by any theory, the present disclosure contemplates that the red bean extract has a high potassium content, which can promote urination. In some embodiments, the present disclosure's red bean extract comprises at least about 50, 100, 150, 200, 250, 300, 350, 400, or 450 mg/100 g of potassium, or any range defined by two foregoing endpoints, such as: 50 to 450 mg/100 g, 100 to 450 mg/100 g, 150 to 450 mg/100 g, 200 to 450 mg/100 g, or 250 to 450 mg/100 g of potassium.

In many embodiments, the red bean extract further comprises a polyphenol compound, which provides an additional effect of antioxidation, elimination of free radicals, prevention of arteriosclerosis, anti-aging, and/or cancer prevention. In some specific embodiments, the polyphenolic compounds may include, but are not limited to, catechin glucoside, rutin, catechin, proanthocyanidins, or combinations thereof, wherein those polyphenol compounds might, individually or in sum, comprises at least about 2, 3, 4, 5, 6, or 7 mg/g, or any range defined by two foregoing endpoints, such as: 2 to 7 mg/g, 2 to 6 mg/g, 2 to 5 mg/g, 2 to 4 mg/g, 3 to 7 mg/g, 3 to 6 mg/g, 3 to 5 mg/g, or 3 to 4 mg/g of the red bean extract.

In some embodiments, the red bean extract might be extracted from various kinds of red beans and is not limited to a certain kind, for example, a species derived from Japan, including Erimosyozu, Kitano-otome, Shumari, Sahorosyozu, Akanedanagon, Hokutodainagon, and Toyomidainagon; or from China, including Hebei Azuki, Liaoning Azuki, Shanxi Azuki, and Shaanxi Azuki. In some other embodiments, the red bean extract is extracted from a Japanese species of red bean. In some specific embodiments, the red bean extract is an Erimosyozu red bean seed coat extract. In many embodiments, the red bean extract has an antioxidation effect, which, determined by using a Trolox equivalent antioxidant capacity assay, equal at least about 5, 10, 15, 20, 25, 30, 35 μmol Trolox/g antioxidation effect, or any range defined by two foregoing endpoints, such as:5 to 35 μmol Trolox/g, 5 to 30 μmol Trolox/g, 5 to 25 μmol Trolox/g, 5 to 20 μmol Trolox/g, 5 to 15 μmol Trolox/g, 5 to 10 μmol Trolox/g, 10 to 35 μmol Trolox/g, 10 to 30 μmol Trolox/g, 10 to 25 μmol Trolox/g, 10 to 20 μmol Trolox/g, 10 to 15 μmol Trolox/g, 15 to 35 μmol Trolox/g, 15 to 30 μmol Trolox/g, 15 to 25 μmol Trolox/g, 15 to 20 μmol Trolox/g, 20 to 35 μmol.

Botanical Extract. As described above, the composition of the present disclosure comprises a botanical extract, which comprises at least about 1, 2, 3, 4, 5, 6, 7, 8, 9, 10 wt %, or any range defined by two foregoing endpoints, such as: 1 to 10 wt %, 1 to 8 wt %, 1 to 5 wt %, 1 to 3 wt %, 1 to 10 wt %, 2 to 8 wt %, 2 to 5 wt %, 2 to 3 wt %, 3 to 10 wt %, 3 to 8 wt %, 3 to 5 wt %, 5 to 10 wt %, or 5 to 8 wt % of the present disclosure's composition. In some embodiments, the botanical extract comprises an Apiaceae Carum carvi extract, a Spiraea ulmaria extract, a Paullinia cupana extract, a Solidago virgaurea extract, a Foeniculum vulgare Mill extract, and a Taraxacum officinale Weber extract. Without wishing to be bound by any theory, the present disclosure contemplates that any or a combination of the botanical extracts above is capable of inhibiting the reabsorption of sodium ions, thereby increasing urination and sodium excretion. Furthermore, it is believed that the botanical extracts above, alone or in combination, can be helpful in terms of regulating blood vessel constriction, promoting renal blood flow, and improving kidney filtration efficiency, as well as increasing calcium ion absorption, which further increases urination.

In some embodiments, the Apiaceae Carum carvi extract comprises 30 to 40 wt %, 32 to 42 wt %, 32 to 38 wt %, 32 to 36 wt %, or 34 to 36 wt % of the botanical extract. In many embodiments, the Apiaceae Carum carvi extract is extracted from the seed of an Apiaceae Carum carvi extract. In many specific embodiments, the Apiaceae Carum carvi extract is prepared by a method comprising: grinding a seed of an Apiaceae Carum carvi into powder, mixing the powder with water to obtain a mixture, drying the mixture, thereby obtaining the Apiaceae Carum carvi extract. In some embodiments, the method further comprises, before drying, filtering the mixture to remove solid substances therein. The drying can be performed by using spray-drying, freeze-drying, or vacuum drying. In many embodiments, the method further comprises, before drying, sterilizing the mixture, for example, by pasteurization.

In many embodiments, the Spiraea ulmaria extract comprises 25 to 35 wt %, 28 to 35 wt %, 30 to 35 wt %, or 32 to 35 wt % of the botanical extract. In some embodiments, the Spiraea ulmaria extract is extracted from the corolla of Spiraea ulmaria. In a specific embodiment, the Spiraea ulmaria extract is prepared by a method comprising: mixing a corolla of Spiraea ulmaria with water to obtain a mixture, and drying the mixture, thereby obtaining the Spiraea ulmaria extract. In some embodiments, the method further comprises, before drying, filtering the mixture to remove solid substances therein. In some specific embodiments, the drying can be performed by using spray-drying, freeze-drying, or vacuum drying. In many embodiments, the method further comprises, before drying, sterilizing the mixture, for example, by pasteurization.

In many embodiments, the Paullinia cupana extract comprises 25 to 35 wt %, 28 to 35 wt %, 30 to 35 wt %, or 31 to 35 wt % of the botanical extract. In some embodiments, the Paullinia cupana extract is extracted from the seed of Paullinia cupana. In a specific embodiment, the Paullinia cupana extract is prepared by a method comprising: mixing a seed of Paullinia cupana with water to obtain a mixture, and drying the mixture, thereby obtaining the Paullinia cupana extract. In some embodiments, the method further comprises, before drying, filtering the mixture to remove solid substances therein. In some specific embodiments, the drying can be performed by using spray-drying, freeze-drying, or vacuum drying. In many embodiments, the method further comprises, before drying, sterilizing the mixture, for example, by pasteurization.

In many embodiments, the Solidago virgaurea extract comprises 0.1 to 5 wt %, 0.5 to 5 wt %, 1 to 5 wt %, or 1 to 3 wt % of the botanical extract. In some embodiments, the Solidago virgaurea extract is extracted from a stem of a Solidago virgaurea. In a specific embodiment, the Solidago virgaurea extract is prepared by a method comprising: mixing a stem of a Solidago virgaurea with water to obtain a mixture, and drying the mixture, thereby obtaining the Solidago virgaurea extract. In some embodiments, the method further comprises, before drying, filtering the mixture to remove solid substances therein. In some specific embodiments, the drying can be performed by using spray-drying, freeze-drying, or vacuum drying. In many embodiments, the method further comprises, before drying, sterilizing the mixture, for example, by pasteurization.

In some embodiments, the Foeniculum vulgare Mill extract comprises 0.1 to 1 wt %, 0.5 to 1 wt %, 0.1 to 0.8 wt %, or 0.1 to 0.5 wt %. In many embodiments, the Foeniculum vulgare Mill extract is extracted from the fruit of Foeniculum vulgare Mill. In a specific embodiment, the Foeniculum vulgare Mill extract is prepared by a method comprising: mixing a fruit of Foeniculum vulgare Mill with water to obtain a mixture, and drying the mixture, thereby obtaining the Foeniculum vulgare Mill extract. In some embodiments, the method further comprises, before mixing with water, grinding the fruit of the Foeniculum vulgare Mill. In some embodiments, the method further comprises, before drying, filtering the mixture to remove solid substances therein. In some specific embodiments, the drying can be performed by using spray-drying, freeze-drying, or vacuum drying. In many embodiments, the method further comprises, before drying, sterilizing the mixture, for example, by pasteurization.

In many embodiments, the Taraxacum officinale Weber extract comprises 0.1 to 1 wt %, 0.5 to 1 wt %, 0.1 to 0.8 wt %, or 0.1 to 0.5 wt % of the botanical extract. In some embodiments, the Taraxacum officinale Weber extract is extracted from the leaf of Taraxacum officinale Weber. In a specific embodiment, the Taraxacum officinale Weber extract is prepared by a method comprising: mixing a leaf of Taraxacum officinale Weber with water to obtain a mixture, and drying the mixture, thereby obtaining the Taraxacum officinale Weber extract. In some embodiments, the method further comprises, before drying, filtering the mixture to remove solid substances therein. In some specific embodiments, the drying can be performed by using spray-drying, freeze-drying, or vacuum drying. In many embodiments, the method further comprises, before drying, sterilizing the mixture, for example, by pasteurization.

In addition to the extraction steps described in the methods above for preparing the respective botanical extract, as the dried botanical extract might be in a powder form, in some embodiments, the powder can be sieved before use to prevent the extract (i.e., the powder) from clumping. The sieving process is also helpful for ensuring an even distribution of the powder and, therefore, facilitates mixing the powder with other ingredients. In some embodiments, the extract mentioned herein can be further mixed with an excipient, such as, but not limited to, a food thickener (e.g., maltodextrin, cyclodextrin, or dextrin), to increase the volume thereof, thereby facilitating the processing and manufacturing of the product. In some embodiments, before mixing the plant or portions thereof with an extraction solvent (e.g., water), the plant or portions thereof to be extracted can be cut or ground into an appropriate size. Besides, in some embodiments, the method for preparing the extracts might further comprise, after mixing the plant or portions thereof to be extracted with water, heating the mixture to increase extraction efficiency and outcome, but the present disclosure is not limited to such approaches.

Grape Leaf Extract. In some embodiments, the present disclosure further comprises a grape leaf extract, which comprises at least 0.1, 0.2, 0.3, 0.4, 0.5, 0.6, 0.7, 0.8, 0.9, or 1 wt %, or any range defined by two foregoing endpoints, such as: 0.1 to 1 wt %, 0.1 to 0.8 wt %, 0.1 to 0.5 wt %, 0.1 to 0.3 wt %, 0.2 to 1 wt %, 0.2 to 0.8 wt %, 0.2 to 0.5 wt %, 0.2 to 0.3 wt %, 0.3 to 1 wt %, 0.3 to 0.8 wt %, 0.3 to 0.5 wt %, 0.5 to 1 wt %, or 0.5 to 0.8 wt % of the present disclosure's composition. In some specific embodiments, the grape leaf extract is extracted from the leaf of a grape (e.g., Vitis vinifera L). Without wishing to be bound by any theory, the present disclosure contemplates that the grape leaf extract can improve lower limb edema caused by chronic venous insufficiency. In many embodiments, the grape leaf extract comprises a flavonoid.

In a specific embodiment, the grape leaf extract is an aqueous extract, which can be prepared by a method comprising: mixing a grape leaf with water to obtain a mixture, condensing the mixture, and drying the mixture, thereby obtaining a grape leaf extract. In some embodiments, the condensing reduces at least 10%, 20%, 30%, 40%, 50%, 60%, 70%, 80%, or 90% of the volume of the mixture, which can be achieved by reducing the water content of the mixture. In some specific embodiments, the drying can be performed by using spray-drying, freeze-drying, or vacuum drying. In some specific embodiments, the method further comprises, before drying, homogenizing the condensed mixture. In addition, the method might further comprise mixing the mixture with an excipient during homogenization. In some specific embodiments, the method further comprises grinding the dried grape leaf extract and may further mix it evenly.

Adlay extract. In many embodiments, the present disclosure further comprises an adlay extract, which comprises 0.1, 0.2, 0.3, 0.5, 0.8, 1, 3, or 5 wt %, or any range defined by two foregoing endpoints, such as: 0.1 to 5 wt %, 0.1 to 3 wt %, 0.1 to 1 wt %, 0.1 to 0.5 wt %, 0.1 to 0.3 wt %, 0.5 to 5 wt %, 0.5 to 3 wt %, 0.5 to 1 wt %, 1 to 5 wt %, or 1 to 3 wt % of the present disclosure's composition. Without wishing to be bound by any theories, the present disclosure contemplates that the adlay extract comprises potassium, magnesium, calcium, vitamin B, coixenolide, adlay esters, essential amino acids, dietary fiber, or a combination thereof, thereby promoting urination, lowering blood lipids, and reducing blood sugar. In a specific embodiment, the adlay extract is an aqueous extract, which can be prepared by a method comprising: mixing a raw adlay with water to obtain a mixture, condensing the mixture, and drying the mixture, thereby obtaining the adlay extract. In some embodiments, the condensing reduces at least 10%, 20%, 30%, 40%, 50%, 60%, 70%, 80%, or 90% of the volume of the mixture, which can be achieved by reducing the water content of the mixture. In some specific embodiments, the drying can be performed by using spray-drying, freeze-drying, or vacuum drying.

Preparation of the Present Disclosure's Composition. The composition of the present disclosure can be prepared by more than one method as long as the prepared composition comprises the components in the amounts as described in the present disclosure. In some specific embodiments, a method for preparing the present disclosure's composition comprises: obtaining the required extracts, for example, obtaining 14.29 to 57.14 gram of the botanical extract (which comprises the Apiaceae Carum carvi extract, the Spiraea ulmaria extract, the Paullinia cupana extract, the Solidago virgaurea extract, the Foeniculum vulgare Mill extract, and the Taraxacum officinale Weber extract), 2.86 to 5.71 gram of the Haematococcus pluvialis extract (which comprises 1 to 10 wt % of astaxanthin), 1.43 to 7.14 gram of the Erimosyozu red bean seed coat extract, 0 to 5.71 gram of the grape leaf extract, 0 to 20 gram of the adlay extract; mixing the extracts with an excipient evenly to obtain a raw material composition of around 1000 grams. The raw material composition can then be formulated into a desired formulation for consumers. In some embodiments, the extracts can be mixed with an excipient respectively and then mixed with one another.

In some embodiments, the composition can be formulated as an oral composition. In many embodiments, the oral composition can be, but is not limited to, a powder, granule, tablet, capsule, solution, suspension, or a combination thereof. In many embodiments, the present disclosure's composition can further comprise a food-grade excipient, including but not limited to a carrier, binder, thickener, diluent, disintegrant, filler, glidant, lubricant, coloring agent, preservative, sweetener, surfactant, solvent, coating agent, or a mixture thereof

### Methods

Another aspect of the present disclosure provides a method for increasing urination. The method comprises administering a composition of the present disclosure to a subject. In some embodiments, the subject is diagnosed or self-diagnosed with edema (e.g., lower limb edema), or the subject is diagnosed or self-diagnosed as likely to benefit from increased urination and is not limited to having shown edema symptoms. In many specific embodiments, the subject is a mammal, including but not limited to human, dog, cat, horse, cattle, monkey, rabbit, mouse, or sheep.

In many embodiments, the subject is administered an effective amount of the composition. In many specific embodiments, the effective amount is to administer at least about 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20 grams per 60 Kg-Body Weight of the present disclosure's composition, or any range defined by two foregoing endpoints, such as: 2 to 20 grams, 2 to 18 grams, 2 to 16 grams, 2 to 14 grams, 2 to 10 grams, 2 to 8 grams, 2 to 6 grams, 2 to 4 grams, 3 to 20 grams, 3 to 18 grams, 3 to 16 grams, 3 to 14 grams, 3 to 10 grams, 3 to 8 grams, 3 to 6 grams, 3 to 4 grams, 4 to 20 grams, 4 to 18 grams, 4 to 16 grams, 4 to 14 grams, 4 to 10 grams, 4 to 8 grams, 4 to 6 grams, 5 to 20 grams, 5 to 18 grams, 5 to 16 grams, 5 to 14 grams, 5 to 10 grams, 5 to 8 grams, 5 to 6 grams, 10 to 20 grams, 10 to 18 grams, 10 to 16 grams, or 10 to 14 grams. In some embodiments, the effective amount refers to an amount for daily administration.

In some embodiments, the effective amount can be achieved by multiple administrations. For instance, in some embodiments, the administration is performed 1, 2, 3, 4, 5, 6, 7, 8, 9, or 10 times, or more, or any range defined by two foregoing endpoints, such as: 1 to 10 times, 1 to 8 times, 1 to 6 times, 1 to 4 times, 3 to 10 times, 3 to 8 times, 3 to 6 times, or 3 to 4 times. In many embodiments where the administration is performed at least 2 times, the at least 2 times of administrations has an interval of about 1 hour, 4 hours, 8 hours, 12 hours, 16 hours, 24 hours, 2 days, 4 days, 1 week, 2 weeks, 1 month, 3 months, or 6 months, or any range defined by two foregoing endpoints, such as: 1 to 4 hours, 1 to 8 hours, 1 to 24 hours, 24 hours to 2 days, 2 to 4 days, 1 week to 1 month, 1 to 3 months, 1 to 6 months, or 3 to 6 months. In many embodiments where the administration is performed at least 3 times, the intervals between any two consecutive administrations may be the same or different. For example, the first administration might be separated from the second administration for 4 hours, while the second administration might be separated from the third administration for 8 hours. In many embodiments, the administration is performed orally.

### Additional Definitions

As described herein, the term “comprising,” “including,” or “having” refers to the presence of the specified features, values, steps, operations, members, components, and/or combinations thereof but does not exclude the possibility of the presence of other features, values, steps, operations, members, components, and/or combinations thereof. As described herein, only the phrase “consisting of” may be interpreted as a closed-ended transition, and only the term “consisting essentially of” may be interpreted as a semi-closed transition.

As described herein, an “effective amount” of a composition refers to an amount that can achieve the described clinical outcome or therapeutic effect within a specific desired timeframe. An effective amount may be achieved through multiple administrations. If the subject receiving the administration or treatment with the composition has been confirmed to have the described disease or symptom, the effective amount may be referred to as a “therapeutically effective amount.” For a subject at risk of developing the disease or symptom (e.g., recurrence), the effective amount administered to the individual may be referred to as a “prophylactically effective amount.”

As described herein, the term “administration” refers to the act of providing the composition to a subject, where the subject may self-administer or be administered the composition. The term “administration” used in the present disclosure is not limited to a specific route, quantity, frequency, or method through which the subject receives the composition.

Example 1: The efficacy of the present disclosure's composition in increasing urination.

The experiment demonstrates the efficacy of an exemplary composition of the present disclosure in increasing urination (i.e., diuretic effect). The exemplary composition comprised 1.5 wt % of an ELMREAL® composition, 0.3 wt % of a Haematococcus pluvialis extract, 0.5 wt % of a Erimosyozu red bean seed coat extract, 0.2 wt % of a grape leaf extract (Nutrifin Vitis™), 1 wt % of a mixed extract of adzuki bean and adlay (CoreBean™; MCB group); wherein the remaining percentage of the composition is an excipient (e.g., a flavoring agent). The ELMREAL® composition comprised an Apiaceae Carum carvi extract (Carum carvi), a Spiraea ulmaria extract, a Paullinia cupana extract, a Solidago virgaurea extract, a Taraxacum officinale Weber extract, and a Foeniculum vulgare Mill extract.

Experiment Design. A total of 16 rats (Wistar, 8 weeks old) were used in this experiment. Those 16 rats were randomly assigned to an experimental group and a control group. The experiment was divided into a first phase and a second phase with a one-week interval, and urine samples were collected from 0 to 6 hours and 6 to 24 hours of each phase. The first phase was designed to determine a benchmark urination of the rats. Firstly, the rats were fasted for 16 hours prior to the experiment. After the 16-hour fasting period, their body weights were measured, and each rat was administered with PBS equivalent to 4% of its body weight, followed by a normal diet. The rats were then individually housed to ensure independent collection of urine samples and accurate measurement of urination

Based on the urination recorded in the first phase, the three rats with the lowest urination in both the experimental and control groups were excluded (i.e., these 6 rats did not participate in the second phase of the experiment). This design was to eliminate individuals with abnormally low urination in order to minimize the impact of individual variability on the experimental results. Subsequently, prior to the second phase of the experiment, the remaining rats were fasted for 16 hours. After the fasting period, their body weights were measured. The rats in the experimental group (5 rats) were administered the exemplary composition disclosed herein at a dose of 725 mg/kg body weight. This dose was calculated based on the human recommended intake of 117 mg/kg body weight (1×dose for rats is defined as 6.2 times the recommended human daily intake per kg body weight). Simultaneously, the control group rats (5 rats) were administered with PBS equivalent to 4% of their body weight. The rats were then individually housed to ensure independent collection of urine samples and accurate recording of urination.

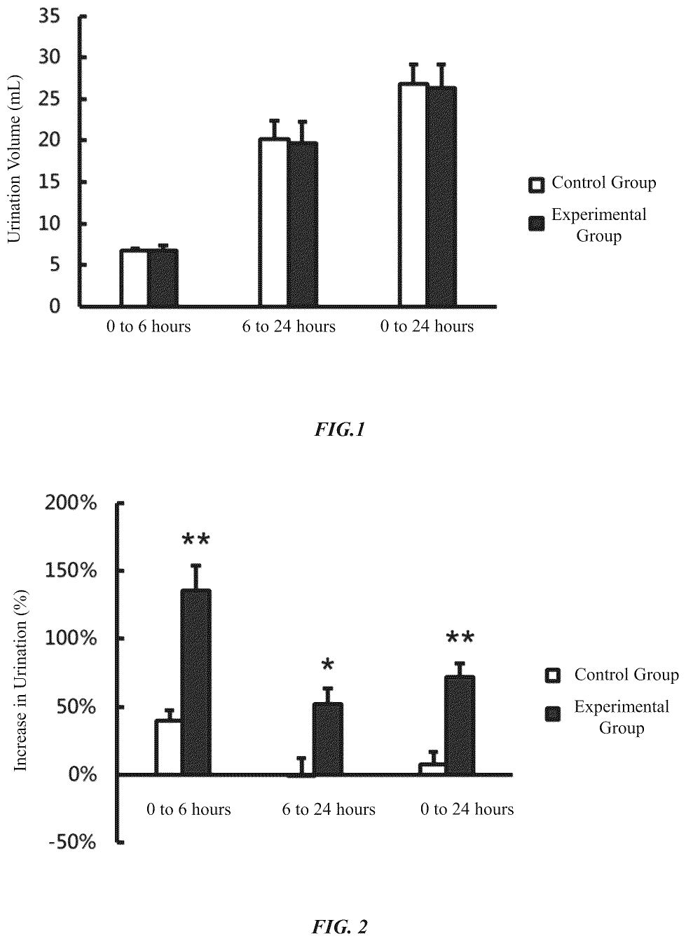

Results: According to the record of the first phase, there was no significant difference in body weight between the experimental rats and the control rats. The urination of the experimental rats and the control rats in the 0 to 6 hours period was about 6.68±0.62 mL and 6.69±0.34 mL respectively; in the 6 to 24 hours period, the urination was about 19.64±2.68 mL and 20.20±2.21 mL, respectively; in the 0 to 24 hours period, it was about 26.31±2.86 mL and 26.89±2.26 mL. That said, the rats from the two groups did not show significant differences in urination in the 0 to 6 hours period and the 6 to 24 hours period, and total amounts of urination collected in the 24 hours were also quite similar (FIG. 1). This record verified that there wasn't obvious individual variability between the randomly assigned experiment group and control group.

Then, as noted above, in order to highlight the experimental results, three rats of both the experimental and control groups with the lowest urination amount in the first phase were excluded. Additionally, after excluding the data from these six rats in the first phase, the body weight and urination of the experimental and control groups were recalculated to assess whether the removal of the lowest-urine-output rats affected the baseline comparison between the two groups. The results indicated that there were no significant differences in body weight or urination between the two groups. This finding suggests that excluding the three lowest-urination rats from each group did not compromise the representativeness of the experimental and control groups; that is, there was still no significant individual variability between the groups.

According to the record of the second phase and using the record of the first phase as a benchmark, the changes in urination of the experimental rats and the control rats in the 0 to 6 hours period were about +136.04±0.18% and +39.86±0.08% respectively; in the 6 to 24 hours period, the urination changes were about +51.73±0.12% and −0.95±0.13%; in the 0 to 24 hours period, the urination changes were about +72.25±0.10% and +7.62±0.09% (FIG. 2). The difference between the experimental rats and the control rats was significant. This data verified that the exemplary composition of the present disclosure was able to increase urination significantly. Besides, these statistical results also show that the efficacy of the present disclosure's exemplary composition was immediate, as it increased urination in a short period (0 to 6 hours).

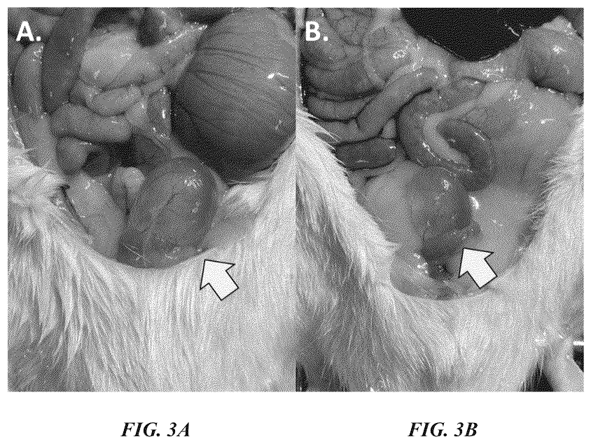

Lastly, all rats used in this experiment were sacrificed, and their bladders were excised for examination to assess any abnormalities. No pathological changes were observed in the bladders of the experimental group rats, and no significant differences were noted between the control and experimental groups (FIG. 3). These results support that the exemplary composition disclosed herein did not cause bladder damage.

### EMBODIMENTS

Embodiment 1: A composition for increasing urination, comprising: 1 to 10 wt % of a botanical extract, which comprises an Apiaceae Carum carvi extract, a Spiraea ulmaria extract, a Paullinia cupana extract, a Solidago virgaurea extract, a Foeniculum vulgare Mill extract, and a Taraxacum officinale Weber extract; 0.1 to 1 wt % of a Haematococcus pluvialis extract; and 0.1 to 1 wt % of a red bean extract.

Embodiment 2: The composition for increasing urination of Embodiment 1, wherein the red bean extract is a seed coat extract.

Embodiment 3: The composition for increasing urination of Embodiment 2, wherein the seed coat extract is prepared by mixing a red bean with water to obtain a mixture, heating the mixture, drying the heated mixture, thereby obtaining the seed coat extract.

Embodiment 4: The composition for increasing urination of Embodiment 3, wherein the mixture was boiled for 10 to 60 minutes.

Embodiment 5: The composition for increasing urination of Embodiment 3 or Embodiment 4, wherein heating is performed at 90 to 120 degrees Celsius.

Embodiment 6: The composition for increasing urination of any one of Embodiments 3 to 5, wherein before drying the heated mixture, condensing the heated mixture.

Embodiment 7: The composition for increasing urination of any one of Embodiments 3 to 6, wherein the drying is performed using spray-drying.

Embodiment 8: The composition for increasing urination of any one of Embodiments 1 to 7, further comprising 0.1 to 5 wt % of an adlay extract.

Embodiment 9: The composition for increasing urination of Embodiment 8, wherein the adlay extract is an aqueous extract.

Embodiment 10: The composition for increasing urination of any one of Embodiments 1 to 9, wherein the Haematococcus pluvialis extract comprises 1 to 10 wt % of astaxanthin.

Embodiment 11: The composition for increasing urination of Embodiment 10, wherein the astaxanthin comprises 20% to 100% of left-handed astaxanthin.

Embodiment 12: The composition for increasing urination of any one of Embodiments 1 to 11, further comprising 0.1 to 1 wt % of a grape leaf extract.

Embodiment 13: The composition for increasing urination of Embodiment 12, wherein the grape leaf extract is obtained from a leaf of Vitis vinifera.

Embodiment 14: The composition for increasing urination of Embodiment 12 or Embodiment 13, wherein the grape leaf extract is an aqueous extract.

Embodiment 15: The composition for increasing urination of any one of Embodiments 1 to 14, wherein the botanical extract comprises 30 to 40 wt % of the Apiaceae Carum carvi extract, 25 to 35 wt % of the Spiraea ulmaria extract, 25 to 35 wt % of the Paullinia cupana extract, 0.1 to 5 wt % of the Solidago virgaurea extract, 0.1 to 1 wt % of the Foeniculum vulgare Mill extract, and 0.1 to 1 wt % of the Taraxacum officinale Weber extract.

Embodiment 16: The composition for increasing urination of Embodiment 15, wherein the Apiaceae Carum carvi extract comprises 34 wt % of the botanical extract.

Embodiment 17: The composition for increasing urination of Embodiment 15 or Embodiment 16, wherein the Spiraea ulmaria extract comprises 32 wt % of the botanical extract.

Embodiment 18: The composition for increasing urination of any one of Embodiments 15 to 17, wherein the Paullinia cupana extract comprises 31 wt % of the botanical extract.

Embodiment 19: The composition for increasing urination of any one of Embodiments 15 to 18, wherein the Solidago virgaurea extract comprises 1.2 wt % of the botanical extract.

Embodiment 20: The composition for increasing urination of any one of Embodiments 15 to 19, wherein the Foeniculum vulgare Mill extract comprises no more than 1 wt % of the botanical extract.

Embodiment 21: The composition for increasing urination of any one of Embodiments 15 to 20, wherein the Taraxacum officinale Weber extract comprises no more than 1 wt % of the botanical extract.

Embodiment 22: The composition for increasing urination of any one of Embodiments 1 to 21, wherein the Apiaceae Carum carvi extract is an aqueous extract.

Embodiment 23: The composition for increasing urination of any one of Embodiments 1 to 22, wherein the Apiaceae Carum carvi extract is a seed extract.

Embodiment 24: The composition for increasing urination of any one of Embodiments 1 to 23, wherein the Spiraea ulmaria extract is an aqueous extract.

Embodiment 25: The composition for increasing urination of any one of Embodiments 1 to 24, wherein the Spiraea ulmaria extract is a corolla extract.

Embodiment 26: The composition for increasing urination of any one of Embodiments 1 to 25, wherein the Paullinia cupana extract is an aqueous extract.

Embodiment 27: The composition for increasing urination of any one of Embodiments 1 to 26, wherein the Paullinia cupana extract is a seed extract.

Embodiment 28: The composition for increasing urination of any one of Embodiments 1 to 27, wherein the Solidago virgaurea extract is an aqueous extract.

Embodiment 29: The composition for increasing urination of any one of Embodiments 1 to 28, wherein the Solidago virgaurea extract is a stem extract.

Embodiment 30: The composition for increasing urination of any one of Embodiments 1 to 29, wherein the Foeniculum vulgare Mill extract is an aqueous extract.

Embodiment 31: The composition for increasing urination of any one of Embodiments 1 to 30, wherein the Foeniculum vulgare Mill extract is a fruit extract.

Embodiment 32: The composition for increasing urination of any one of Embodiments 1 to 31, wherein the Taraxacum officinale Weber extract is an aqueous extract.

Embodiment 33: The composition for increasing urination of any one of Embodiments 1 to 32, wherein the Taraxacum officinale Weber extract is a leaf extract.

Embodiment 34: The composition for increasing urination of any one of Embodiments 1 to 33, being an oral composition.

Embodiment 35: The composition for increasing urination of any one of Embodiments 1 to 34, further comprising an excipient.

Embodiment 36: The composition for increasing urination of Embodiment 35, wherein the excipient comprises a carrier, binder, thickener, diluent, disintegrant, filler, glidant, lubricant, coloring agent, preservative, sweetener, surfactant, solvent, coating agent, or a mixture thereof.

Embodiment 37: A composition for increasing urination, comprising 1 to 10 wt % of a botanical extract, which comprises an Apiaceae Carum carvi extract, a Spiraea ulmaria extract, a Paullinia cupana extract, a Solidago virgaurea extract, a Foeniculum vulgare Mill extract, and a Taraxacum officinale Weber extract; 0.001 to 0.1 wt % of astaxanthin; and 0.1 to 1 wt % of a red bean extract.

Embodiment 38, The composition for increasing urination of claim 37, wherein the astaxanthin is purified from Haematococcus pluvialis, Adonis aestivalis, Euphasia supurba, salmon, red snapper, or a mixture thereof.

Embodiment 39: The composition for increasing urination of Embodiment 37 or Embodiment 38, wherein the astaxanthin comprises 20% to 100% of left-handed astaxanthin.

Embodiment 40: The composition for increasing urination of any one of Embodiments 37 to 39, further comprising a Haematococcus pluvialis extract, wherein the Haematococcus pluvialis extract comprises the astaxanthin, and the astaxanthin comprises 1 to 10 wt % of the Haematococcus pluvialis extract.

Embodiment 41: The composition for increasing urination of Embodiment 40, wherein the Haematococcus pluvialis extract comprises 0.1 to 1 wt % of the composition.

Embodiment 42: The composition for increasing urination of any one of Embodiments 37 to 42, wherein the red bean extract is a seed coat extract.

Embodiment 43: The composition for increasing urination of Embodiment 42, wherein the seed coat extract is prepared by: mixing a red bean with water to obtain a mixture, heating the mixture, drying the heated mixture, thereby obtaining the seed coat extract.

Embodiment 44: The composition for increasing urination of Embodiment 43, wherein the mixture was boiled for 10 to 60 minutes.

Embodiment 45: The composition for increasing urination of Embodiment 43 or Embodiment 44, wherein heating is performed at 90 to 120 degrees Celsius.

Embodiment 46: The composition for increasing urination of any one of Embodiments 43 to 45, wherein before drying the heated mixture, condensing the heated mixture.

Embodiment 47: The composition for increasing urination of any one of Embodiments 43 to 46, wherein the drying is performed using spray-drying.

Embodiment 48: The composition for increasing urination of any one of Embodiments 37 to 47, further comprising 0.1 to 5 wt % of an adlay extract.

Embodiment 49: The composition for increasing urination of Embodiment 48, wherein the adlay extract is an aqueous extract.

Embodiment 50: The composition for increasing urination of any one of Embodiments 37 to 49, further comprising 0.1 to 1 wt % of a grape leaf extract.

Embodiment 51: The composition for increasing urination of Embodiment 50, wherein the grape leaf extract is obtained from a leaf of Vitis vinifera.

Embodiment 52: The composition for increasing urination of Embodiment 50 or Embodiment 51, wherein the grape leaf extract is an aqueous extract.

Embodiment 53: The composition for increasing urination of any one of Embodiments 37 to 52, wherein the botanical extract comprises 30 to 40 wt % of the Apiaceae Carum carvi extract, 25 to 35 wt % of the Spiraea ulmaria extract, 25 to 35 wt % of the Paullinia cupana extract, 0.1 to 5 wt % of the Solidago virgaurea extract, 0.1 to 1 wt % of the Foeniculum vulgare Mill extract, and 0.1 to 1 wt % of the Taraxacum officinale Weber extract.

Embodiment 54: The composition for increasing urination of Embodiment 53, wherein the Apiaceae Carum carvi extract comprises 34 wt % of the botanical extract.

Embodiment 55: The composition for increasing urination of Embodiment 53 or Embodiment 54, wherein the Spiraea ulmaria extract comprises 32 wt % of the botanical extract.

Embodiment 56: The composition for increasing urination of any one of Embodiments 53 to 55, wherein the Paullinia cupana extract comprises 31 wt % of the botanical extract.

Embodiment 57: The composition for increasing urination of any one of Embodiments 53 to 56, wherein the Solidago virgaurea extract comprises 1.2 wt % of the botanical extract.

Embodiment 58: The composition for increasing urination of any one of Embodiments 53 to 57, wherein the Foeniculum vulgare Mill extract comprises no more than 1 wt % of the botanical extract.

Embodiment 59: The composition for increasing urination of any one of Embodiments 53 to 58, wherein the Taraxacum officinale Weber extract comprises no more than 1 wt % of the botanical extract.

Embodiment 60: The composition for increasing urination of any one of Embodiments 37 to 59, wherein the Apiaceae Carum carvi extract is an aqueous extract.

Embodiment 61: The composition for increasing urination of any one of Embodiments 37 to 60, wherein the Apiaceae Carum carvi extract is a seed extract.

Embodiment 62: The composition for increasing urination of any one of Embodiments 37 to 61, wherein the Spiraea ulmaria extract is an aqueous extract.

Embodiment 63: The composition for increasing urination of any one of Embodiments 37 to 62, wherein the Spiraea ulmaria extract is a corolla extract.

Embodiment 64: The composition for increasing urination of any one of Embodiments 37 to 63, wherein the Paullinia cupana extract is an aqueous extract.

Embodiment 65: The composition for increasing urination of any one of Embodiments 37 to 64, wherein the Paullinia cupana extract is a seed extract.

Embodiment 66: The composition for increasing urination of any one of Embodiments 37 to 65, wherein the Solidago virgaurea extract is an aqueous extract.

Embodiment 67: The composition for increasing urination of any one of Embodiments 37 to 66, wherein the Solidago virgaurea extract is a stem extract.

Embodiment 68: The composition for increasing urination of any one of Embodiments 37 to 67, wherein the Foeniculum vulgare Mill extract is an aqueous extract.

Embodiment 69: The composition for increasing urination of any one of Embodiments 37 to 68, wherein the Foeniculum vulgare Mill extract is a fruit extract.

Embodiment 70: The composition for increasing urination of any one of Embodiments 37 to 69, wherein the Taraxacum officinale Weber extract is an aqueous extract.

Embodiment 71: The composition for increasing urination of any one of Embodiments 37 to 70, wherein the Taraxacum officinale Weber extract is a leaf extract.

Embodiment 72: The composition for increasing urination of any one of Embodiments 37 to 71, being an oral composition.

Embodiment 73: The composition for increasing urination of any one of Embodiments 37 to 75, further comprising an excipient.

Embodiment 74: The composition for increasing urination of Embodiment 73, wherein the excipient comprises a carrier, binder, thickener, diluent, disintegrant, filler, glidant, lubricant, coloring agent, preservative, sweetener, surfactant, solvent, coating agent, or a mixture thereof.

Embodiment 75: A method for increasing urination, comprising administering a composition of any one of Embodiments 1 and 74 to a subject.

Embodiment 76, The method of Embodiment 75, wherein an effective amount of the composition is 2 to 20 g/per 60 Kg body weight.

Embodiment 77, The method of Embodiment 76, wherein an effective amount of the composition is 2 to 10 g/per 60 Kg body weight.

Embodiment 78, The method of Embodiment 77, wherein an effective amount of the composition is 4 to 8 g/per 60 Kg body weight.

Embodiment 79: The method of any one of Embodiments 75 to 78, wherein the administering is performed orally.

Embodiment 80: The method of any one of Embodiments 75 to 79, wherein the subject is administered with the composition at least twice.

Embodiment 81: The method of any one of Embodiments 75 to 80, wherein the subject is administered with the composition at least twice per day.

Embodiment 82: A use of a mixture for preparing a composition for increasing urination, wherein the mixture comprises a botanical extract, a Haematococcus pluvialis extract, and a red bean extract; wherein the botanical extract comprises a Apiaceae Carum carvi extract, a Spiraea ulmaria extract, a Paullinia cupana extract, a Solidago virgaurea extract, a Foeniculum vulgare Mill extract, and a Taraxacum officinale Weber extract, and the composition is according to any one of Embodiments 1 to 74.

What is claimed is

1. A composition for increasing urination, comprising: 1 to 10 wt % of a botanical extract, which comprises an Apiaceae Carum carvi extract, a Spiraea ulmaria extract, a Paullinia cupana extract, a Solidago virgaurea extract, a Foeniculum vulgare Mill extract, and a Taraxacum officinale Weber extract; 0.1 to 1 wt % of a Haematococcus pluvialis extract; and 0.1 to 1 wt % of a red bean extract.

2. The composition for increasing urination of claim 1, wherein the red bean extract is a seed coat extract, which is prepared by mixing a red bean with water to obtain a mixture, heating the mixture, drying the heated mixture, thereby obtaining the seed coat extract.

3. The composition for increasing urination of claim 1, further comprising 0.1 to 5 wt % of an adlay extract.

4. The composition for increasing urination of claim 1, wherein the Haematococcus pluvialis extract comprises 1 to 10 wt % of astaxanthin.

5. The composition for increasing urination of claim 1, further comprising 0.1 to 1 wt % of a grape leaf extract.

6. The composition for increasing urination of claim 1, wherein the botanical extract comprises 30 to 40 wt % of the Apiaceae Carum carvi extract, 25 to 35 wt % of the Spiraea ulmaria extract, 25 to 35 wt % of the Paullinia cupana extract, 0.1 to 5 wt % of the Solidago virgaurea extract, 0.1 to 1 wt % of the Foeniculum vulgare Mill extract, and 0.1 to 1 wt % of the Taraxacum officinale Weber extract.

7. The composition for increasing urination of claim 6, wherein the botanical extract comprises 34 wt % of the Apiaceae Carum carvi extract, 32 wt % of the Spiraea ulmaria extract, 31 wt % of the Paullinia cupana extract, 1.2 wt % of the Solidago virgaurea extract, 1 wt % of the Foeniculum vulgare Mill extract, and 1 wt % of the Taraxacum officinale Weber extract.

8. The composition for increasing urination of claim 1, wherein the Apiaceae Carum carvi extract is a seed extract, the Spiraea ulmaria extract is a corolla extract, the Paullinia cupana extract is a seed extract, the Solidago virgaurea extract is a stem extract, the Foeniculum vulgare Mill extract is a fruit extract, and/or the Taraxacum officinale Weber extract is a leaf extract.

9. The composition for increasing urination of claim 1, further comprising an excipient, comprising a carrier, binder, thickener, diluent, disintegrant, filler, glidant, lubricant, coloring agent, preservative, sweetener, surfactant, solvent, coating agent, or a mixture thereof.

10. A composition for increasing urination, comprising 1 to 10 wt % of a botanical extract, which comprises an Apiaceae Carum carvi extract, a Spiraea ulmaria extract, a Paullinia cupana extract, a Solidago virgaurea extract, a Foeniculum vulgare Mill extract, and a Taraxacum officinale Weber extract; 0.001 to 0.1 wt % of astaxanthin; and 0.1 to 1 wt % of a red bean extract, wherein the astaxanthin is purified from Haematococcus pluvialis, Adonis aestivalis, Euphasia supurba, salmon, red snapper, or a mixture thereof.

11. The composition for increasing urination of claim 10, further comprising a Haematococcus pluvialis extract, wherein the Haematococcus pluvialis extract comprises the astaxanthin, and the astaxanthin comprises 1 to 10 wt % of the Haematococcus pluvialis extract.

12. The composition for increasing urination of claim 10, wherein the red bean extract is a seed coat extract, which is prepared by mixing a red bean with water to obtain a mixture, heating the mixture, drying the heated mixture, thereby obtaining the seed coat extract.

13. The composition for increasing urination of claim 10, further comprising 0.1 to 5 wt % of an adlay extract, and/or 0.1 to 1 wt % of a grape leaf extract.

14. The composition for increasing urination of claim 10, wherein the botanical extract comprises 30 to 40 wt % of the Apiaceae Carum carvi extract, 25 to 35 wt % of the Spiraea ulmaria extract, 25 to 35 wt % of the Paullinia cupana extract, 0.1 to 5 wt % of the Solidago virgaurea extract, 0.1 to 1 wt % of the Foeniculum vulgare Mill extract, and 0.1 to 1 wt % of the Taraxacum officinale Weber extract.

15. The composition for increasing urination of claim 14, wherein the botanical extract comprises 34 wt % of the Apiaceae Carum carvi extract, 32 wt % of the Spiraea ulmaria extract, 31 wt % of the Paullinia cupana extract, 1.2 wt % of the Solidago virgaurea extract, 1 wt % of the Foeniculum vulgare Mill extract, and 1 wt % of the Taraxacum officinale Weber extract.

16. The composition for increasing urination of claim 10, wherein the Apiaceae Carum carvi extract is a seed extract, the Spiraea ulmaria extract is a corolla extract, the Paullinia cupana extract is a seed extract, the Solidago virgaurea extract is a stem extract, the Foeniculum vulgare Mill extract is a fruit extract, and/or the Taraxacum officinale Weber extract is a leaf extract.

17. The composition for increasing urination of claim 10, further comprising an excipient, comprising a carrier, binder, thickener, diluent, disintegrant, filler, glidant, lubricant, coloring agent, preservative, sweetener, surfactant, solvent, coating agent, or a mixture thereof.

18. A method for increasing urination, comprising administering an effective amount of a composition of claim 1 to a subject.

19. The method of claim 18, wherein the effective amount is 2 to 20 g/per 60 Kg body weight.

20. The method of any one of claim 18, wherein the subject is administered with the composition at least twice.
